(19) United States
(12) Patent Application Publication
(10) Pub. No.: US 2025/0339682 A1
(43) Pub. Date: Nov. 6, 2025

(54) RESPIRATION PROMOTING APPARATUS

(71) Applicant: STIMIT AG, Biel/Bienne (CH)

(72) Inventors: Christian BRUDERER, Winterthur (CH); Hans MESSERLI, Kriegstetten (CH)

(21) Appl. No.: 18/869,443

(22) PCT Filed: Jun. 1, 2023
(86) PCT No.: PCT/EP2023/064655

§ 371 Date: Nov. 26, 2024

(30) Foreign Application Priority Data
Jun. 1, 2022 (CH) 000659/2022

(51) Int. Cl. A61N 1/36; A61N 1/04

(52) U.S. Cl. CPC A61N 1/3601; A61N 1/0456; A61N 1/0472; A61N 1/3603

(57) ABSTRACT

A respiration promoting apparatus (1) to coordinately stimulate two Phrenic nerves at a neck of a patient for activating a diaphragm of the patient, comprises a first field generator (21), a second field generator (21) and a support structure (3). The first field generator (21) is configured to generate a first spatial field and to be positioned at the patient to stimulate a first Phrenic nerve of the two Phrenic nerves of the patient by means of the first spatial field. The second field generator (22) is configured to generate a second spatial field and to be positioned at the patient to stimulate a second Phrenic nerve of the two Phrenic nerves of the patient by means of the second spatial field. The support structure (3) has an arm holder (33), a first arm (31) and a second arm (32). The first arm (31) and the second arm (32) extend from the arm holder (33). The first field generator (21) is mounted to the first arm (31) and the second field generator (22) is mounted to the second arm (32). The support structure (3) is configured to position the arm holder (33) above or behind a head the patient such that the first arm (31) and the second arm (32) laterally extend along the head towards the neck of the patient.

### TECHNICAL FIELD

The present invention relates to a respiration promoting apparatus. Such respiration promoting apparatus comprising a first field generator configured to be positioned to stimulate a first Phrenic nerve of a patient, a second field generator configured to to stimulate a second Phrenic nerve of the patient, and a support structure to which the first and second field generators are mounted mounted can be used for to coordinately stimulate the two Phrenic nerves at a neck of the patient for activating a diaphragm of the patient.

### BACKGROUND ART

In medicine, it is known that for many purposes it is beneficial to activate a target tissue of a patient using stimulation by electro-magnetic, electric or magnetic fields. For achieving such activation of tissues in a patient's body, it is known to directly stimulate the tissue or to indirectly activate the tissue via stimulation of specific parts of the neural system. For example, the target tissue being a muscular tissue can be activated by providing electric pulses directly to the muscular tissue or to nerves associated to the muscular tissue.

In critical care units or other departments of hospitals, it may be desired to activate the diaphragm for ventilating patients, typically in combination with mechanical ventilation, in order to prevent drawbacks of disuse of the diaphragm. It was shown that disuse atrophy of diaphragm muscle fibres occurs already in the first 18-69 hours of mechanical ventilation, and the muscle fibre cross-sections decreased by more than 50% in this time. Thus, it is aimed to activate the diaphragm repeatedly while the patient is given artificial or mechanical respiration such that the functioning of the diaphragm can be upheld, or to activate the diaphragm at least during the weaning period to support effective restoration of independent respiratory function.

It is known that the diaphragm can be activated by stimulating the two Phrenic nerves, e.g., at the neck of a patient. In this context, US 2016/0310730 A1 describes an apparatus for reducing ventilation induced diaphragm disuse in a patient receiving ventilation support from a mechanical ventilator. The apparatus includes an electrode array of first and second types and comprises a plurality of electrodes configured to stimulate a Phrenic nerve of the patient. At least one controller identifies a type of electrode array and generates a stimulus signal for stimulating a Phrenic nerve of the patient based upon the identity of the electrode type. Such electrode-based stimulation is not very robust to patient movements or relocations, and the possible stimulation depth can be significantly limited by bones or fatty tissue. Also, electrode stimulation is reported to be more painful for the patient than electro-magnetic stimulation.

In one example, the electrode arrays are configured as surface electrode arrays embedded in adhesive electrode patches to be placed on each side of a patient's neck near the areas where the Phrenic nerves are located. Thus, the electrode arrays are electrically and mechanically coupled to the skin of the patient independent from each other. Therefore, the electrode arrays cover portions of a patient's surface and may interfere with other applications required for the treatment of the patient like for example the intubation into the trachea. In case the electrode arrays need to be taken off and replaced at a later point in time, the system needs to be fully re-calibrated according the new locations of the electrode arrays to avoid undesired co-stimulation of tissue surrounding the Phrenic nerves.

In practice, also electro-magnetic stimulators are used to activate a target tissue, which are based on the principle of electro-magnetic induction. A strong current pulse (typically a monophasic or biphasic current pulse) flows through a coil winding, which produces a strong, transient magnetic field. The current pulses cause a changing magnetic field that for example alters according to the phases of the current pulses. The changing magnetic field induces a corresponding electric field, which in turn depolarizes neuronal membranes, leading to action potentials through one or more nerves. Coil windings are usually designed towards generating electric field distribution curves of the induced field, often having an electric field peak (area with maximum electric field strength) or an electric field area, which is stronger than other fields in other areas. Thus, an electric field distribution curve can generate activation pulses, which are effective periodically at time intervals, and electric field peaks or strong field areas may alternate with low field areas in the distribution curve.

However, when two target nerves inside the body in a close distance to each other, like the two Phrenic nerves, shall be stimulated simultaneously, the coil winding systems in today's stimulators used for electro-magnetic stimulation have significant limitations. De-central coils have been designed for this purpose producing de-centralized fields and have been described for instance in DE 10 2007 013531 A1. However, typically two separate stimulator devices are necessary, the electromagnetic fields of the two devices can interfere with each other, and body constraints may not allow positioning coils windings of the two devices in parallel to make use of the de-centralized design. Especially in the neck region, positioning coil windings longitudinal to the neck would force the user to choose significantly smaller coil winding sizes because the chin and chest constrain space for the coils to be placed.

For addressing such downsides of the configurations know in the art, WO 202/074453 A1 suggests to provide a bracket structure coupled to a first field generator and a second field generator. The bracket structure as well as the first and second field generators each have a forward face configured to be positioned at the patient. In particular, the first and second field generators are positioned at a neck of the patient where they can access the Phrenic nerves and the bracket structure is positioned on the sternum of the patient. The bracket structure is adjustable such that positions and orientations of the first field generator and of the second field generator are adapted as the need may be. In particular, by changing a configuration for adjusting the bracket structure the position of the first field generator and the second field generator relative to the patient and relative to each other can be adapted to specific treatment requirements of a specific patient for example by choosing differing distances between the field generators and a patient's body. Further, the orientation of first and second field generator relative to each other and the orientation of the field generators relative to the patient can be adapted for example by choosing differing angle adjustments for the first and the second field generators.

However, even though the bracket structure of the prior art allows for a comparably stable arrangement on the patient's body and for appropriately adjusting location and orientation of the field generators, locating the bracket structure on the sternum may be deteriorate access to the patient. For example, when in addition to stimulation of the Phrenic nerves the patient is mechanically ventilated and/or tracheotomy has to be applied, the bracket structure being also in front of the patient might be hindering or cause cumbersome situations. Also, cables of the field generators may additionally complicate the arrangement of all required or beneficial therapeutic means to be applied to the patient. Still further, as all the components in front of the patient can physically or operationally disturb each other, most appropriate positioning of the field generator may be impeded or even made impossible.

Therefore, there is a need for a respiration promoting apparatus allowing for efficient stimulation of both Phrenic nerves, overcome space constraints, avoid co-stimulation effects of tissue in the vicinity of the Phrenic nerves, are simple to apply at a patient as well as convenient and pain-free for the patient.

### DISCLOSURE OF THE INVENTION

According to the invention this need is settled by a respiration promoting apparatus as it is defined by the features of independent claim 1. Preferred embodiments are subject of the dependent claims.

In particular, the invention deals with a respiration promoting apparatus to coordinately stimulate two Phrenic nerves at a neck of a patient for activating a diaphragm of the patient. The respiration promoting apparatus comprises a first field generator, a second field generator and a support structure. The first field generator is configured to generate a first spatial field and to be positioned at the patient to stimulate a first Phrenic nerve of the two Phrenic nerves of the patient by means of the first spatial field. The second field generator is configured to generate a second spatial field and to be positioned at the patient to stimulate a second Phrenic nerve of the two Phrenic nerves of the patient by means of the second spatial field. The support structure has an arm holder, a first arm and a second arm. The first arm and the second arm extend from the arm holder. The first field generator is mounted to the first arm and the second field generator is mounted to the second arm. The support structure is configured to position the arm holder above or behind a head of the patient such that the first arm and the second arm laterally extend along the head towards the neck of the patient.

The term “position” as used herein refers to a location and orientation. Changing the position of an element involves either relocating the element, reorienting the element or a combination thereof. If an element or component is positioned to be capable of doing something, it advantageously is located and orientated to achieve the respective function. For example, the field generator being positioned to stimulate a Phrenic nerve may relate to being located and oriented such that the phrenic nerve is within the spatial field generated by the field generator.

The term “positioned at a body” or, similarly, “holding at a body” relates correspondingly to be located and oriented at the body. In connection with the field generator these terms can relate to being physically in contact with a body of the patient and particularly to the neck or in close distance to it. The location and orientation of the field generator or a component of it can thereby be predefined or distinct to be appropriate for activating the diaphragm by stimulating the Phrenic nerves. In order to be configured for being positioned at an appropriate position, the field generator can be formed to be suited to the respective position. For example, it can be formed in in correspondence to a neck of the patient such that it can conveniently be positioned at the neck, e.g., for stimulating one of the Phrenic nerves. Since the diaphragm of the patient is intended to be activated, e.g. for ventilation, indirect activation may be induced by positioning the first and second field generators such that one or both Phrenic nerve(s) are located in the spatial field generated by the field generator thereby stimulating the Phrenic nerve(s).

The term “spatial field” as used herein relates to any field allowing stimulation of the Phrenic nerves of the patient. It may particularly involve an electric field or an electro-magnetic field.

The spatial field can be configured to have a targeted shape. Such targeted shape can be achieved by providing a locally constrained, targeted electric or electro-magnetic field, e.g., having a peak. It can be adapted to be active in a target area being the Phrenic nerve area that shall be stimulated with the spatial fields of the first and second field generators, which can be for example achieved by the peak of the spatial field (focality area). The targeted shape can generally be any shape of the spatial field or component thereof that allows to stimulate the Phrenic nerves effectively while minimizing other undesired co-stimulation effects of surrounding, above-lying or close-by tissues or nerves. A peak shape is such example, because it maximizes effects in the focality area and minimizes effects outside this focality area.

For generating the spatial field, the field generator can comprise a coil design or coil unit. Thereby, the term “coil design” can be or comprise at least two coil windings or at least one cone shaped or otherwise curved or bulged coil, or at least one cylindrical or otherwise non-flat coil, or at least one small coil, i.e. a coil sufficiently small to generate a sharp electro-magnetic field such as a coil having a diameter of 9 cm or less.

By means of the support structure being configured to position the arm holder above or behind the head of the patient, the arms can particularly latero-facially extend from the arm holder to the neck. Such arrangement allows for preventing that any component of the respiration promoting apparatus is arranged in front of the patient. In particular, the front side of the neck as well as the mouth and the nose of the patient may be kept accessible or free. Thus, space constraints can be overcome and a sophisticated stimulation of both Phrenic nerves can be achieved. Also, co-stimulation effects of tissue in the vicinity of the Phrenic nerves may be avoided and application of the apparatus can be specifically simple, convenient and low pain for the patient.

The support structure may be configured to flexibly adapt the position of the field generators. Thereby, the first and second field generators may be positioned relative to their respective Phrenic nerve independent from each other.

In particular, the first arm and the second arm preferably are manually adjustable relative to the arm holder to individually position the first field generator and the second field generator in a target position at the neck of the patient. The term “target position” as used herein relates to a location and orientation of the first or second field generator being appropriate for stimulating the associated Phrenic nerve at the neck of the patient. In particular, in the target position, the respective field generator can generate the spatial field which reaches the associated Phrenic nerve, advantageously in a specific manner preventing undesired stimulation of other tissue of the neck of the patient. Like this, the apparatus can conveniently be individualized to the respective patient such that a specifically and efficiently stimulate the Phrenic nerves.

The respiration promoting apparatus preferably comprises a locking mechanism to block the first arm and the second arm in the target positions. Like this, the support structure can be fixed to the conditions of the individual patient and the apparatus can conveniently be kept in the individualized configuration.

Thereby, the locking mechanism comprises a first lever element at the first arm to operate the locking mechanism with respect to the first arm and a second lever element at the second arm to operate the locking mechanism with respect to the second arm. By having the two lever arms, the single arms can individually by adjusted and locked in the respective target position.

The locking mechanism thereby preferably is configured to allow adjustment of the first arm when the first lever element is activated, to block adjustment of the first arm when the first lever element is not activated, to allow adjustment of the second arm when the second lever element is activated, and to block adjustment of the second arm when the second lever element is not activated. In connection with the first and second lever elements, the term “activate” can relate to pressing or pushing the lever element or a portion thereof. This allows for a particularly efficient and convenient adjustment of the support structure and, more specifically, of its arms.

The locking mechanism preferably comprises a first tensioning arrangement blocking the first arm in the target position when being tensioned, and a second tensioning arrangement blocking the second arm in the target position when being tensioned. Such tensioning elements allow to firmly block the arms of the support structure.

Thereby, the first tensioning arrangement preferably comprises a first resilient member blocking the first arm in the target position when being tensioned and the second tensioning arrangement preferably comprises a second resilient member blocking the second arm in the target position when being tensioned. Like this, the locking mechanism can be in a tensioned and blocked in a null or non-activated state.

Activation of the first lever element preferably releases the first resilient member of the first tensioning arrangement and activation of the second lever element preferably releases the second resilient member of the first tensioning arrangement.

Preferably, the first arm, the second arm and the arm holder are configured such that a length of the first arm and a length of the second arm are adjustable. When being provided with the locking mechanism such length adjustments may be limited to a state where the locking mechanism is not blocking. By having lengths adjustable arms, the support structure may efficiently be adjustable to varying sizes of different patients.

Preferably, each of the first arm and the second arm comprises a front arm portion, an intermediate arm portion, a back arm portion, a front joint member connecting the front arm portion to the intermediate arm portion, and a back joint member connecting the intermediate arm portion to the back arm portion. Such two joint embodiment of the arms allows for providing sufficient flexibility to accurately adjust the arms. Also, such two joint embodiment may provide a comparably high robustness also in context of blocking a specific configuration. Each of the first arm and the second arm preferably comprises less than six joint members. By limiting the maximum number of joint member per arm, the force required for blocking and/or unblocking the single arms can be kept in range which may be manually handled by a majority of users. In other words, it can be prevented that a force to applied for blocking and/or unblocking is inconveniently high.

Thereby, the support structure preferably comprises two shifter members each connecting one of the back arm portions of the first arm and the second arm to the arm holder. Such shifter members allow for individually adjust a length of the arms, which can be beneficial for adjusting the arms.

Each of the front joint members and the back joint members preferably comprises a ball joint. Such ball joints may provide an advantageous flexibility and robustness in a comparably simple construction.

Preferably, the support structure comprises a body portion and a stand, wherein the arm holder is mounted to the body portion and wherein the body portion is mounted to the stand. By means of the stand, the apparatus can be soundly placed at a target location. The body portion in turn may allow to appropriately position the arm holder such that the arms can advantageously extend and be properly arranged.

Thereby, an angle between the arm holder and the body portion is adjustable. Like this, a tilt of the arm holder and, thus, the arms can be adapted as required.

In order to achieve an appropriate adjustment of the angle, the support structure preferably comprises a hinge member connecting the arm holder to the body portion.

The body portion is releasably mounted to the stand. Like this, the stand can conveniently be placed at the target location in separate step and, afterwards, the body portion can be positioned once the stand the stand is safely arranged.

The stand preferably comprises a head plate configured to accommodate the head of the patient and a beam member extending from head plate. In particular, the beam member can upwardly extend from the head plate, when the head plate is arranged at its target location. By such configuration, the stand can be safely held by the head of the patient at an appropriate position.

Thereby, the body portion preferably is height adjustably mounted to the beam member of the stand. Like this, the given situation of the individual patient can be taken into account when adjusting or configuring the respiration promoting apparatus.

The stand preferably has at least one wing element arranged to laterally fold out from the head plate. Advantageously, the stand has two wing elements configured to be folded out in opposite directions. Such at least one wing element allows for increasing stability of the stand when being placed at the target location.

Preferably, the respiration promoting apparatus comprises a control unit configured to operate the first field generator to stimulate the first Phrenic nerve of the patient and to operate the second field generator to stimulate the second Phrenic nerve of the patient.

Thereby, the support structure preferably comprises a first button at the first arm of the support structure and a second button at the second arm of the support structure, wherein the first button and the second button are interfacing with the control unit such that activation of the first button and/or the second button provides control signals to the control unit. The buttons may be a push button, touch sensors or the like. Such configuration allows for verifying if the location of the individual field generator is appropriate to stimulate the respective Phrenic nerve. In particular, by applying single stimulation pulses, it can be tested if an appropriate reaction is induced by the field generator and, for example, the adjustment of the arm can be blocked once such reaction is identified.

Preferably, the support structure is equipped with a data storage configured to collect data about operation of the first field generator and the second field generator. Such data storage allows for collecting data about the therapy of a patient. At a certain stage or at the end of the therapy a physician can evaluate the induced stimulation and its achieved therapeutic effect.

Thereby, the data about operation of the first field generator and the second field generator preferably comprises any of duration or intensity, or a combination thereof. In particular, the operation duration and the field intensity during therapy may be crucial parameter to be evaluated by the physician.

### BRIEF DESCRIPTION OF THE DRAWINGS

The respiration promoting apparatus according to the invention are described in more detail hereinbelow by way of an exemplary embodiment and with reference to the attached drawings, in which:

FIG. 1 shows a front view of an embodiment of a respiration promoting apparatus according to the invention;

FIG. 2 shows a side view of the respiration promoting apparatus of FIG. 1;

FIG. 3 shows a back view of the respiration promoting apparatus of FIG. 1;

FIG. 4 shows a top view of the respiration promoting apparatus of FIG. 1;

FIG. 5 shows a perspective view of the respiration promoting apparatus of FIG. 1; and

FIG. 6 shows a cross sectional view of the respiration promoting apparatus of FIG. 1.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

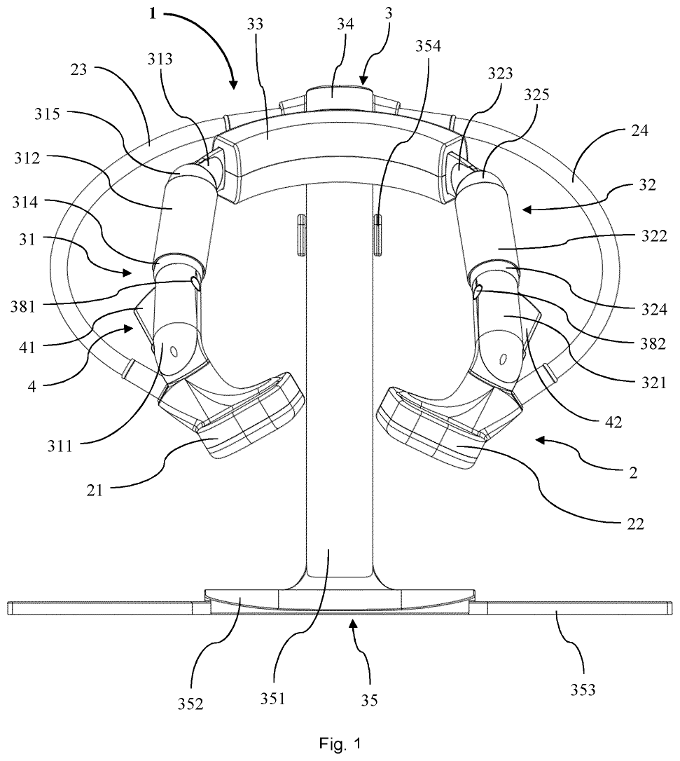

FIG. 1 shows a front view of an embodiment of a respiration promoting apparatus 1 according to the invention. The respiration promoting apparatus 1 is configured to coordinately stimulate two Phrenic nerves at a neck of a patient for activating a diaphragm of the patient.

The respiration promoting apparatus 1 has a pair of field generators 2 comprising a first coil unit 21 as coil design of a first field generator configured to generate a first spatial field, and a second coil unit 22 as coil design of a second field generator configured to generate a second spatial field. The first coil design and particularly its first coil unit 21 is designed to be positioned at the patient to stimulate a first Phrenic nerve of the two Phrenic nerves by means of the first spatial field. Likewise, the second field generator and particularly its second coil unit 22 is designed to be positioned at the patient to stimulate a second Phrenic nerve of the two Phrenic nerves by means of the second spatial field. The first field generator comprises a first power and data cable 23, and the second field generator comprises a second power and data cable 24.

The respiration promoting apparatus further comprises a support structure 3 having an arm holder 33, a first arm 31 extending from the arm holder 33 and a second arm 32 extending from the arm holder 33. The first arm 31 has a first front arm portion 311 equipped with a first button 381, a first intermediate arm portion 312 and a first back arm portion 313. The first coil unit 21 is fixedly mounted to the first front arm portion 311. The first front arm portion 311 is connected to the first intermediate arm portion 312 via a first front ball joint 314 as front joint member, and the first intermediate arm portion 312 is connected to the first back arm portion 313 via a first back ball joint 315 as back joint member. The first back arm portion 313 is length adjustably mounted to a left lateral end of the arm holder 33.

The second arm 32 is embodied in a more or less mirror-inverted manner to the first arm 31. It has a second front arm portion 321 equipped with a second button 382, a second intermediate arm portion 322 and a second back arm portion 323. The second coil unit 22 is fixedly mounted to the second front arm portion 321. The second front arm portion 321 is connected to the second intermediate arm portion 322 via a second front ball joint 324 as front joint member, and the second intermediate arm portion 312 is connected to the second back arm portion 323 via a second back ball joint 325 as back joint member. The second back arm portion 323 is length adjustably mounted to a right lateral end of the arm holder 33.

The support structure 3 further comprises a body portion 34 releasably mounted to a stand 35. The body portion 34 is connected to the first coil unit 21 by the first power and data cable 23, and to the second coil unit 22 by the second power and data cable 24.

The stand 35 has a beam member 351 equipped with a height adjustment switch 354, a flat head plate 352 and two wing elements 353. The beam member 351 upwardly extends from the head plate 352 and the wing elements 353 laterally extend from the head plate 352 to the left and to the right, respectively.

The respiration promoting apparatus 1 further comprises a locking mechanism 4. The locking mechanism 4 has a first lever element 41 arranged at the first front arm portion 311 and a second lever element 42 arranged at the second front arm portion 321.

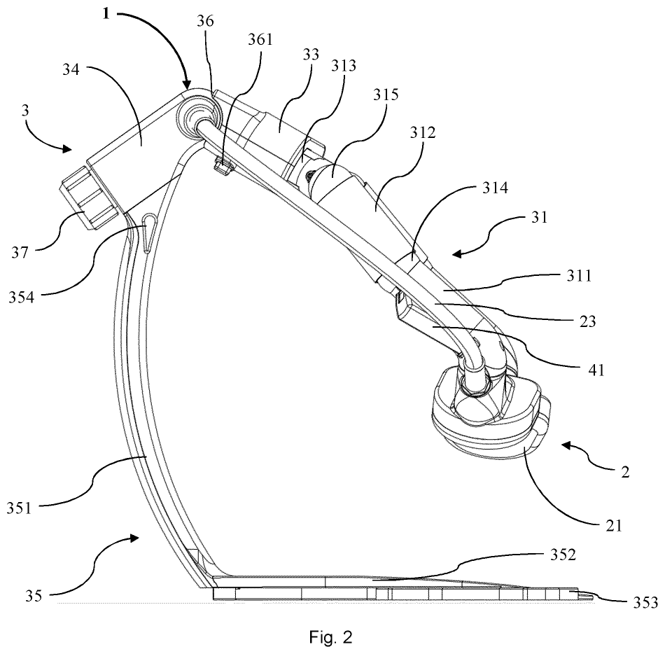

In FIG. 2 the respiration promoting apparatus 1 is shown from aside. Thereby, it can be seen that the beam member 351 is slightly bent and together with the head plate 352 forms a seat for the head of the patient, when the patient is lying. In fact, the head of the patient properly positioned on the head plate 352 effects a stable and safe arrangement of the respiration promoting apparatus 1. Thereby, the arm holder 33 is located above the head of the lying patient and the first and second arms 31, 32 latero-facially extend along the head towards the neck of the patient. The first coil unit 21 can then properly by positioned at a right side of the neck such that the right-sided Phrenic nerve can be stimulated and the second coil unit 22 can the properly by positioned at a left side of the neck such that the left-sided Phrenic nerve can be stimulated.

The beam member 351 has two portions allowing height adjustment. In particular, by switching the height adjustment switch 354 into an open position, the two portions of the beam member 351 can be up-and downwardly shifted relative to each other. One the height adjustment switch 354 is switched back into its basic closed position shown in FIG. 2, the two portions are blocked to each other and a height of the beam member 351 is set.

To properly adjust an angle of the arm holder 33 together with the arms 31, 32 and the coil units 21, 22, the arm holder 33 is mounted to the body portion 34 via a hinge member 36. In particular, the hinge member 36 comprises a hinge de-lock button 361 pressing of which sets free the hinge member 36 such that free rotation of the arm holder 33 relative to the body portion 34 in a predefined range is possible. By letting the hinge de-lock button 361 go again, the hinge member 36 is blocked in the respective position. Like this, the hinge member 36 is configured to allow flexible and patient specific adjustment of the angle between the body portion 34 and the arm holder 33.

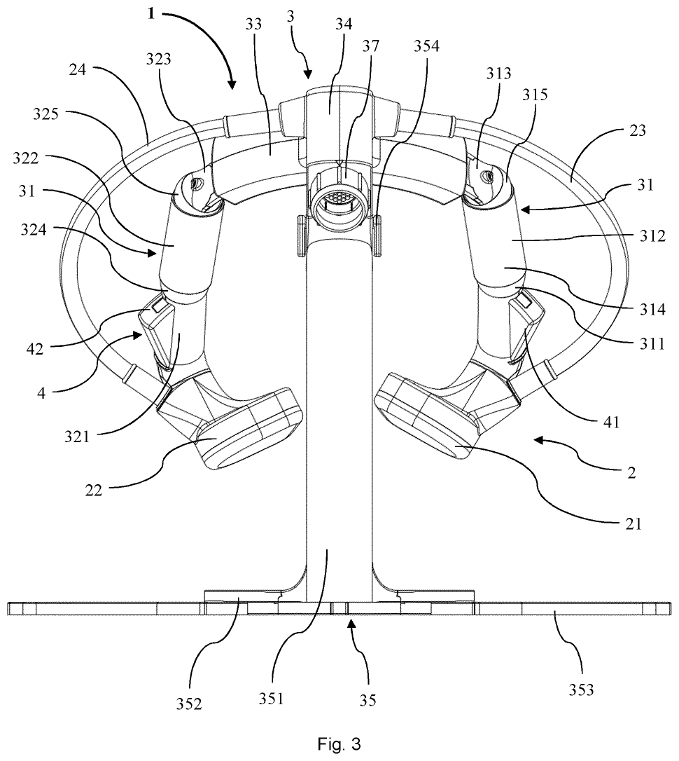

As can be seen in FIG. 2 and particularly in FIG. 3, the body portion 34 is equipped with a control unit connector 37. By means of the control unit connector 37, the coil units 21, 22 and components of the support structure 3 can be connected to a control unit of the respiration promoting apparatus 1 (not visible in the Figs.). For example, the control unit provides power and defines intensity and course of operation of the coil units 21, 22. Like this, various sophisticated therapeutic stimulations can be applied.

The body portion 34 is equipped with a data storage (not visible in the Figs.). The data storage is configured to collect data about operation of the coil units 21, 22 such as number of field propagations, field intensities, durations and the like. Like this, the specific therapy applied to the patient can be analyzed. In particular, by means of the data storage, usage and therapeutic application of the respiration promoting apparatus 1 can be monitored and evaluated.

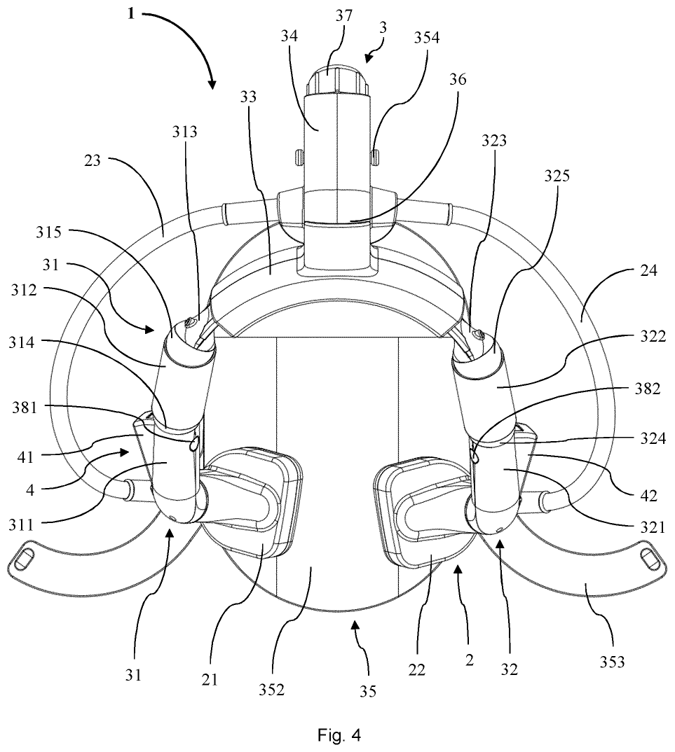

FIG. 4 shows a view top down on the respiration promoting apparatus 1. Thereby, it can be seen that the wing elements 353 of the stand 35 laterally extend from head plate 352 to a left side and to a right side of the head of the patient, respectively. Like this, the head plate 352 contact surface between the respiration promoting apparatus 1 and the structure on which it is positioned can be widened such that stability of the complete respiration promoting apparatus 1 can be increased.

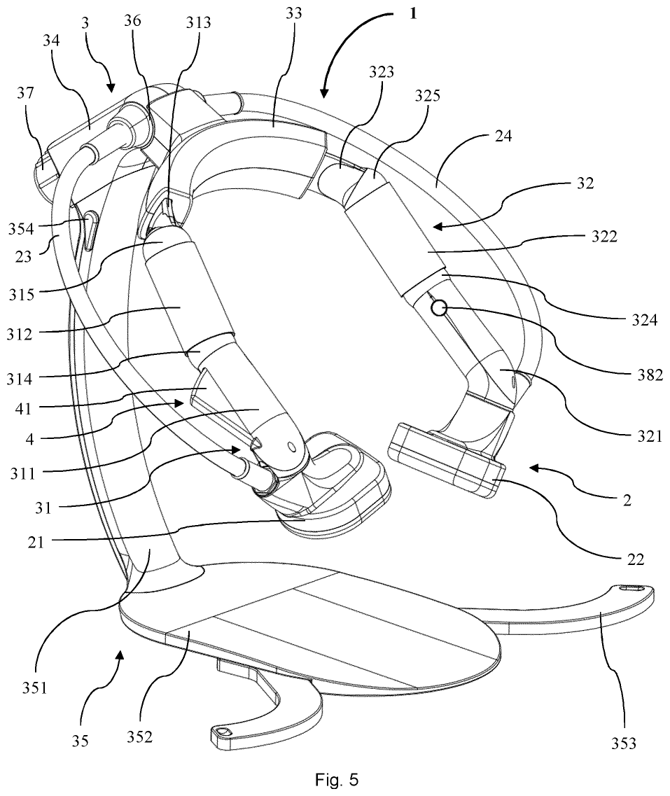

In FIG. 5 a perspective view of the respiration promoting apparatus 1 is shown. The wing elements 353 can pivot relative to the head plate 352 such that they can be folded in and out to be adjusted to the given situation at the place where the respiration promoting apparatus 1 is to be positioned. When not needed, the wing elements 353 can be pivoted completely into the head plate 352.

For being specifically adjusted to the conditions of the patient the arms 31, 32 are manually adjustable relative to the arm holder 33 to individually position the first coil unit 21 and the second coil unit 22 in a target position at the neck of the patient such that the Phrenic nerves can efficiently be stimulated. More specifically, a practitioner can hold the two arms 31, 32 of the support structure at the first front arm portion 311 and the second front arm portion 321 with his two hands. Thereby, the first lever element 41 and/or the second lever element 42 can be pressed or activated such that the lock mechanism 4 is released or loosened. In this state of the lock mechanism 4, the first arm 31 and the second arm 32, respectively, can be freely formed by the degrees of freedom provided by the front joint members 314, 324 and the back joint members 315, 325. Furthermore, lengths of the arms 31, 32 can be set by sliding them relative into or out of the arm holder 33. Like this, the coil units 21, 22 can be sophisticatedly positioned at the neck of the patient.

At the same time, i.e., without requiring to let go the arms 31, 32, the practitioner can access the first button 381 and/or the second button 382 with a thumb. When pressing one of the buttons 381, 382, a signal is transferred to the control unit which in turn activates the associated first or second coil unit 21, 22 to generate a field to stimulate the respective Phrenic nerve. The practitioner can determine if the patient produces an appropriate reaction on the stimulation or if the associated coil unit 21, 22 has to be repositioned. Once the coil units 21, 22 are properly positioned, the practitioner stops pressing or activating the first and second lever elements 41, 42 which causes the lock mechanism to block any further change of the shape of the arms 31, 32. Like this, the individual configuration of the support structure 3 can be set for the patient and the arms 31, 32 are blocked in this target position.

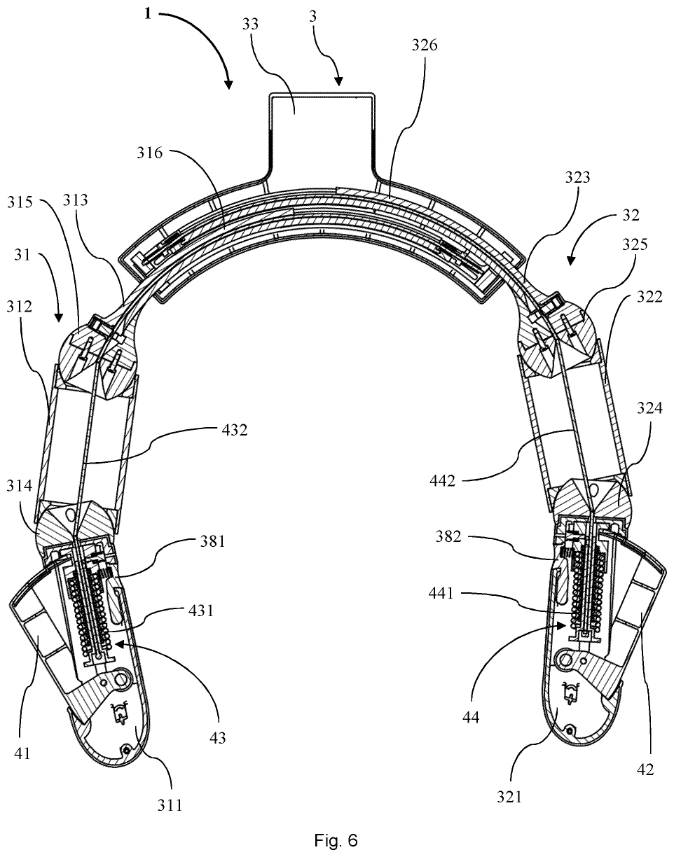

FIG. 6 shows the arms 31, 32 of the support structure 3 in a cross sectional view such that components of the locking mechanism 4 are visible. The first back arm portions 313 of the first arm 31 comprises first shifting members 316 received by a corresponding recess formation of the arm holder 33. Likewise, the second back arm portions 323 of the second arm 32 comprises second shifting members 326 received by a corresponding recess formation of the arm holder 33. Each of the first front joint member 314, the first back joint member 315, the second front joint member 324 and the second back joint member 325 is embodied as ball joint.

The locking mechanism 4 comprises a first tensioning arrangement 43 with a first tensioning cable 432 and a first spring 431 as first resilient member. The first spring 431 is coupled to the first tensioning cable 432 and pretensioned such that a tensile force acts on the first tensioning cable 432 when the first lever element 41 is not activated. In this status, the first front joint member 314 and the first back joint member 315 as well as the first shifting members 316 are fixed such that the length, location and orientation of the first arm 31 is blocked. For adjusting the first arm 31, the first lever element 41 is activated which deforms the first spring 431 against its spring force. Like this, the first tensioning cable 432 is loosened and the first arm can freely be rotated about its joint members 314, 315 and adapted along its shifting members 316.

Similarly as the first tensioning arrangement 43, the locking mechanism 4 additionally comprises a second tensioning arrangement 44 with a second tensioning cable 442 and a second spring 441 as second resilient member. The second spring 441 is coupled to the second tensioning cable 442 and pretensioned such that a tensile force acts on the second tensioning cable 442 when the second lever element 42 is not activated. In this status, the second front joint member 324 and the second back joint member 325 as well as the second shifting members 326 are fixed such that the length, location and orientation of the second arm 32 is blocked. For adjusting the second arm 32, the second lever element 42 is activated which deforms the second spring 441 against its spring force. Like this, the second tensioning cable 442 is loosened and the second arm can freely be rotated about its joint members 324, 325 and adapted along its shifting members 326.

As shown in the Figs., the support structure 3 is configured to position the arm holder 33 above or behind the head of the patient such that the first arm 31 and the second arm 31 laterally extend along the head towards the neck of the patient. Moreover, by means of the first and second arms 31, 32 having portion connected by ball joints, being shiftable relative to the arm holder 33 and being equipped with the locking mechanism 4, the coil units 21, 22 can be moved in all degrees of freedom to be properly positioned at the neck of the patient for efficiently stimulating the Phrenic nerves and, when being in the target position, the arms 31, 32 can be blocked such that the respiration promoting apparatus is set to the individual conditions of the patient.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A respiration promoting apparatus to coordinately stimulate two Phrenic nerves at a neck of a patient for activating a diaphragm of the patient, comprising:
a first field generator configured to generate a first spatial field and configured to be positioned at the patient to stimulate a first Phrenic nerve of the two Phrenic nerves of the patient by means of the first spatial field;
a second field generator configured to generate a second spatial field and configured to be positioned at the patient to stimulate a second Phrenic nerve of the two Phrenic nerves of the patient by means of the second spatial field; and
a support structure having an arm holder, a first arm and a second arm,
wherein the first arm and the second arm extend from the arm holder,
wherein the first field generator is mounted to the first arm and the second field generator is mounted to the second arm, and
wherein the support structure is configured to position the arm holder above and/or behind a head of the patient such that the first arm and the second arm laterally extend along the head towards the neck of the patient.

2. The respiration promoting apparatus of claim 1, wherein the first arm and the second arm are manually adjustable relative to the arm holder to individually position the first field generator and the second field generator in a target position at the neck of the patient.

3. The respiration promoting apparatus of claim 2, comprising a locking mechanism to block the first arm and the second arm in the target positions.

4. The respiration promoting apparatus of claim 3, wherein the locking mechanism comprises a first lever element at the first arm to operate the locking mechanism with respect to the first arm and a second lever element at the second arm to operate the locking mechanism with respect to the second arm.

5. The respiration promoting apparatus of claim 4, wherein the locking mechanism is configured to allow adjustment of the first arm when the first lever element is activated, to block adjustment of the first arm when the first lever element is not activated, to allow adjustment of the second arm when the second lever element is activated, and to block adjustment of the second arm when the second lever element is not activated.

6. The respiration promoting apparatus of claim 3, wherein the locking mechanism comprises a first tensioning arrangement blocking the first arm in the target position when being tensioned, and a second tensioning arrangement blocking the second arm in the target position when being tensioned.

7. The respiration promoting apparatus of claim 6, wherein the first tensioning arrangement comprises a first resilient member blocking the first arm in the target position when being tensioned and the second tensioning arrangement comprises a second resilient member blocking the second arm in the target position when being tensioned.

8. The respiration promoting apparatus of claim 5, wherein activation of the first lever element releases the first resilient member of the first tensioning arrangement and activation of the second lever element releases the second resilient member of the second tensioning arrangement.

9. The respiration promoting apparatus of claim 2, wherein the first arm, the second arm and the arm holder are configured such that a length of the first arm and a length of the second arm are adjustable.

10. The respiration promoting apparatus (1) of claim 2, wherein each of the first arm and the second arm comprises a front arm portion, an intermediate arm portion, a back arm portion, a front joint member connecting the front arm portion to the intermediate arm portion, and a back joint member connecting the intermediate arm portion to the back arm portion.

11. The respiration promoting apparatus of claim 9, wherein the support structure comprises two shifter members each connecting one of the back arm portions of the first arm and the second arm to the arm holder.

12. The respiration promoting apparatus of claim 10, wherein each of the front joint members and the back joint members comprises a ball joint.

13. The respiration promoting apparatus of claim 10, wherein each of the first arm and the second arm comprises less than six joint members.

14. The respiration promoting apparatus of claim 1, wherein the support structure comprises a body portion and a stand, wherein the arm holder is mounted to the body portion and wherein the body portion is mounted to the stand and, wherein an angle between the arm holder and the body portion preferably is adjustable.

15. (canceled)

16. The respiration promoting apparatus of claim 14, wherein support structure comprises a hinge member connecting the arm holder to the body portion.

17. The respiration promoting apparatus of claim 14, wherein the body portion is releasably mounted to the stand.

18. The respiration promoting apparatus of claim 14, wherein the stand comprises a head plate configured to accommodate the head of the patient and a beam member extending from head plate, wherein the body portion preferably is height adjustably mounted to the beam member of the stand.

19. (canceled)

20. The respiration promoting apparatus of claim 18, wherein the stand has at least one wing element arranged to laterally fold out from the head plate.

21. The respiration promoting apparatus of claim 1, comprising a control unit configured to operate the first field generator to stimulate the first Phrenic nerve of the patient and to operate the second field generator to stimulate the second Phrenic nerve of the patient, wherein the support structure preferably comprises a first button at the first arm of the support structure and a second button at the second arm of the support structure, wherein the first button and the second button preferably are interfacing with the control unit such that activation of the first button and/or the second button provides control signals to the control unit.

22. (canceled)

23. The respiration promoting apparatus of claim 1, wherein the support structure is equipped with a data storage configured to collect data about operation of the first field generator and the second field generator, wherein the data about operation of the first field generator and the second field generator preferably comprises any of duration or intensity, or a combination thereof.

24. (canceled)
